(19) United States
(12) Patent Application Publication
(10) Pub. No.: US 2010/0239521 A1
(43) Pub. Date: Sep. 23, 2010

(54) METHOD FOR THE PREPARATION OF MICRO- AND NANO-SIZED CARRIER SYSTEMS FOR THE ENCAPSULATION OF BIOACTIVE SUBSTANCES

(76) Inventor: MOHAMMAD REZA MOZAFARI, TEHRAN (IR)

Correspondence Address: CHOOBIN & CHOOBIN CONSULTANCY L.L.C;Barry Choobin, Green Community, Building 3, Ground Floor, Dubai Investment Park, P.O Box 212880, Dubai, AE

(21) Appl. No.: 12/790,991

(22) Filed: Jun. 1, 2010

(51) Int. Cl. A61K 47/48; C07D 239/553; C07K 5/113; C08F 8/30; C08F 8/14

(52) U.S. Cl. 424/78.26; 424/78.3; 544/313; 514/78; 530/331; 514/21.9; 525/374; 525/384

(57) ABSTRACT

The various embodiments herein provide a method for producing a carrier system for the encapsulation or entrapment of a bioactive agent. According to one embodiment herein, producing a carrier complex of a bioactive ingredient disposed within a carrier material, the method comprising the steps of: (a) providing a complexation zone supplied with an aqueous medium containing the carrier material; (b) simultaneously stirring and heating the aqueous medium; (c) adding the bioactive ingredient to the aqueous medium and maintaining the complexation zone under conditions of temperature effective to facilitate complexation of the bioactive ingredient by the carrier material; and (d) recovering from the complexation zone a carrier complex of the bioactive ingredient.

### SPONSORSHIP STATEMENT

The present invention for international filing is sponsored by The Iranian Nanotechnology initiative Council.

### BACKGROUND

1. Technical Field

The embodiments herein generally relates to the field of novel carrier systems, particularly nano-sized drug carrier systems. The present invention more particularly relates to a new method for the preparation of micro- and nano-sized carrier systems for the encapsulation of bioactive substances.

2. Description of the Related Art

Drug carriers are substances that serve as mechanisms to improve the delivery and the effectiveness of drugs. Drug carriers are used in sundry drug delivery systems such as: controlled-release technology to prolong in vivo drug actions; decrease drug metabolism, and reduce drug toxicity.

Carriers are also used in designs to increase the effectiveness of drug delivery to the target sites in order to improve pharmacological actions.

Encapsulation exclusively refers to a condition in which the bioactive material is completely packaged by the carrier system. Entrapment refers to the association of the bioactive material with the carrier system either internally or externally, or, in the case of lipidic carriers such as liposomes, when the bioactive is located (or is solubilized) within the lipidic phase of the liposomes. Complexation refers to both or each of the encapsulation and entrapment phenomena.

The term “carrier complex” relates to a bioactive ingredient which is surrounded by, bonded to or encapsulated by a carrier material such that the complex remains relatively stable in a number of in vivo and in vitro environments. Such an environment may be the cytosol or bodily fluids. Whilst such a complex may be designed so that it does not produce an antigenic effect upon the delivery of a bioactive ingredient (for example the delivery of DNA to a cell), it may also be designed such that it initiates an antigenic effect on the bioactive ingredient (for example for use as a vaccine). It follows that the term “complexation” is the process of a bioactive ingredient being surrounded by, bonded to or encapsulated by a carrier material.

Temperature and mechanical stirring provide adequate energy for the formation of stable carrier complexes. Formation of carrier systems such as niosomes, liposomes and nanoliposomes is not a spontaneous process and sufficient energy must be put into the system to overcome an energy barrier (Mozafari, M. R., 2010, Nanoliposomes: preparation and analysis. In: Liposomes: Methods and Protocols. Volume 1: Pharmaceutical Nanocarriers. Weissig, V. (Ed.) Humana Press, USA, pp 29-50.).

Hence there is a need of a method that does not require volatile organic solvents during the manufacture of the carrier complexes, also a fast and an efficient method that does not require the carrier materials to be hydrated separately prior to heating.

The above mentioned shortcomings, disadvantages and problems are addressed herein and which will be understood by reading and studying the following specification.

### OBJECTIVES OF THE EMBODIMENTS

The primary object of the embodiments herein is to provide a method of producing a carrier system for the encapsulation or entrapment of a bioactive agent.

Another object of the embodiments herein is to provide a method of producing a carrier complex of a bioactive ingredient disposed within a carrier material.

Another object of the embodiments herein is to provide a method, wherein the method does not require volatile organic solvents to be used in the manufacture of carrier complexes.

Another object of the embodiments herein is to provide a fast and efficient method, wherein the method does not require the carrier materials to be hydrated separately prior to heating.

Another object of the embodiments herein is to provide a method having a duration time of up to 2 hours.

Another object of the embodiments herein is to provide a method, wherein the method is performed in a vessel with a stirring unit disposed in its interior.

Another object of the embodiments herein is the storage of the final product under optimum conditions, such as avoiding light exposure and at ideal temperatures and under an inert atmosphere, such as nitrogen or argon, as appropriate.

These and other objects and advantages of the present invention will become readily apparent from the following detailed description taken in conjunction with the accompanying drawings.

### SUMMARY

The various embodiments herein provide a method of producing a carrier complex of a bioactive ingredient disposed within a carrier material, the method comprises;
(a) providing a complexation zone supplied with an aqueous medium containing the carrier material;
(b) simultaneously stirring and heating the aqueous medium;
(c) adding the bioactive ingredient to the aqueous medium and maintaining the complexation zone under conditions of temperature effective to facilitate complexation of the bioactive ingredient by the carrier material; and
(d) recovering from the complexation zone a carrier complex of the bioactive ingredient.

According to another embodiment of the present invention, a carrier complex of a bioactive ingredient disposed within a carrier material as made according to the method as claimed in claims 1 to 38 and/or made in a reaction vessel depicted in FIG. 2.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 illustrates a flow chart explaining the method of producing a carrier system for the encapsulation or entrapment of a bioactive agent according to one embodiment of the embodiments herein.

FIG. 2 illustrates a schematic representation of a reaction vessel used for the manufacture of carrier complexes.

FIG. 3 illustrates the basic protocol of the method of producing carrier complexes.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a method of producing a carrier system for the encapsulation or entrapment of a bioactive agent, more particularly provide a method of producing a carrier complex of a bioactive ingredient disposed within a carrier material.

According to one embodiment herein, A method of producing a carrier complex of a bioactive ingredient disposed within a carrier material, the method comprising the steps of:

(a) providing a complexation zone supplied with an aqueous medium containing the carrier material;
(b) simultaneously stirring and heating the aqueous medium;
(c) adding the bioactive ingredient to the aqueous medium and maintaining the complexation zone under conditions of temperature effective to facilitate complexation of the bioactive ingredient by the carrier material; and
(d) recovering from the complexation zone a carrier complex of the bioactive ingredient.

In accordance with the present invention, there is provided a method of producing a carrier system of a bioactive ingredient disposed within a carrier material.

Step (c) of the method may be incorporated into step (a) and therefore the bioactive ingredient (such as a pharmaceutical compound) can be incorporated in the initial mixture of the aqueous medium. Alternatively, step (c) may be incorporated into step (b). The aqueous medium of step (b) and/or (c) may be heated to a temperature in the range of 30° C. to 80° C. Preferably, the aqueous medium of step (b) is heated to a temperature in the range of 40° C. to 70° C. Step (c) of the method may be further subjected to a temperature that is less than the temperature of the aqueous medium in step (b) and this may be necessary should the bioactive ingredient be sensitive to high temperatures (such as nucleic acids or amino acids). It will be apparent to one skilled in the art that commonly, steps (a) to (c) will be performed simultaneously, provided that the bioactive ingredient is not sensitive to heat. If the bioactive ingredients are sensitive to heat, then only steps (a) and (b) will be performed simultaneously.

The present invention therefore provides for a method, which does not require volatile organic solvents to be used in the manufacture of carrier complexes Furthermore, the method does not require the carrier materials to be hydrated separately prior to heating and this further allows the method to be fast and efficient.

Preferably, the method has a duration time of up to 2 hours, although it is envisaged that most carrier complexes will be produced to a satisfactory quantity and quality within a 30 minute period.

The carrier complex may comprise a niosome, a micelle, a microsphere, a nanosphere, a nanoparticle, a liposome, a nanoliposome, a vesicular phospholipid gel, an archaeosome or a cochleate.

The carrier material used in the method may be selected from one or more of the following: a polymer, a copolymer, a synthetic polymer-lipid conjugate, a lipid, a phospholipid or a non-ionic surfactant. Examples of carrier material include polymers and copolymers such as poly(DL-lactic acid)-poly(ethylene glycol) (PDLLA-PEG), poly(N-isopropylacrylamide)-b-poly(ethylene glycol) (PNIPAAm-b-PEG), and dextran-hydroxyethyl methacrylate-poly(ethylene glycol) (dexHEMA-PEG), synthetic polymer-lipid conjugates such as poly(ethylene glycol)-phosphatidyl ethanolamine (PEG-PE), poly(ethylene glycol)-distearoyl phosphatidyl ethanolamine (PEG-DSPE), and poly(hydroxyethyl L-asparagine)-N-succinyl-dioctadecylamine (PHEA-DODASuc), lipids and phospholipids such as phosphatidylcholine, phosphatidyl ethanolamine, phosphatidylserine, dipalmitoylphosphatidylcholine, dialkyl-type synthetic surfactants, non-ionic surfactants such as series of Spans (e.g. Span 20, Span 40, Span 60, Span 80) and Tweens (e.g. Tween 20, Tween 60, Tween 80), polyoxyethylene-5-cetyl ether and polyoxyethylene-5-stearyl ether, etc. It will be apparent to one skilled in the art that all of these constituents may be used alone or in combination.

The liposomes of the present invention are formed from liposome-forming lipids. “Liposome-forming lipids” (or “vesicle-forming lipids”) are amphiphilic molecules essentially characterized by a packing parameter of 0.74-1.0, inclusive, or by a lipid mixture having an additive packing parameter (the sum of the packing parameters of each component of the liposome multiplied by the mole fraction of each component) in the range between 0.74 and 1, inclusive.

Further, “liposome-forming lipids” in accordance with the invention are lipids having a glycerol backbone wherein at least one, preferably two, of the hydroxyl groups at the head group is substituted by one or more of an acyl, an alkyl or alkenyl group, a phosphate group, preferably an acyl chain (to form an acyl or diacyl derivative), a combination of any of the above, and/or derivatives of the above, and may contain a chemically reactive group (such as an amine, acid, ester, aldehyde or alcohol) at the headgroup, thereby providing a polar head group. Sphingolipids, and especially sphingomyelins, are good alternatives to glycerophospholipids. Typically, a substituting chain, e.g. an acyl, alkyl and/or alkenyl chain, is between about 14 to about 24 carbon atoms in length, and has varying degrees of saturation, thus resulting in fully, partially or non-hydrogenated liposome-forming lipids. Further, the liposome-forming lipid may be of a natural source, semi-synthetic or a fully synthetic lipid, and may be neutral, negatively or positively charged.

Furthermore, the carrier material may further comprises one or more auxiliary materials and such a material may be selected from one or more of the following: membrane stabilizers, charge modifiers and antioxidants. Examples of auxiliary materials that could be used in conjunction with the method include membrane stabilizers such as charge modifiers/inducers such as dicetylphosphate, phosphatidic acid, stearylamine, ganglioside or the like, antioxidants such as a-tocopherol, any of the group tocopheryl phosphates or the like and divalent cations such as calcium or magnesium. The proportions of these auxiliary materials to the constituents are not critical and the exact proportion will be dependent on the application of the carrier complex, the route of administration and the intended physiological conditions that the complex will be entering. Preferably, the proportion of membrane stabilizers is about 0.1% to about 30% molar ratio of the membrane components. More preferably, the proportion of membrane stabilizers is about 5% to about 20% molar ratio of the membrane components. The charge modifiers may be about 0.1% to about 50% molar ratio of the membrane components, but may preferably be about 10% to about 30% molar ratio of the membrane components.

The aqueous medium may be selected from one or more of the following: water, distilled water or Milli-Q reagent grade water, a physiological saline solution, a buffer solution or an aqueous carbohydrate solution or may be a mixture thereof. Preferably, the pH of the aqueous medium is maintained by a buffer. Such buffers include Tricine buffer, phosphate-buffered saline (PBS), isotonic HEPES buffer, Tris-HCl buffer, lactate buffer, citrate buffer or the like. For the stability of the carrier complexes the pH should preferably be in the range of 3 to 10. Preferably, the pH will be in the range of 6 to 8.

A bioactive ingredient may be selected from one or more of the following: pharmaceutical compounds, pharmaceutically bioactive salts, polysaccharides, nucleic acids, polypeptides, vitamins and antioxidants. There is no limitation on the number of types of pharmaceutical compounds that can be encapsulated/incorporated into the carrier complexes according to the present invention. For example, the bioactive ingredients may be antitumor drugs such as 5-fluorouracil, methotrexate, doxorubicin, etc., antibiotics such as gentamicin, gramicidin S, etc., polysaccharides such as dextran, etc., nucleic acids such as DNA, RNA, siRNA and other polynucleotides, vitamins such as vitamin A, vitamin E, etc., antioxidants such as glutathione, beta carotene, etc. and other general drugs such as sodium salicylate, indomethacin, acetaminophen, etc. Indeed, any bioactive substance for the treatment of disease or conditions could be incorporated into the carrier complex. Furthermore, any substance required for research, such as carboxyfluorescein, fluorescein, sulforhodamine and methylene-blue, could also be incorporated into the carrier complex and may be used for the study (such as characterization or uptake) of drug delivery in vitro for example.

The carrier complexes may be further processed to aid administration of the complexes to the human or animal body and they may additionally be mixed with buffers or other physiologically stable agents to assist administration and/or release of the bioactive ingredients in the complexes.

If the carrier material contains lipids, the temperature in step (b) is more effective to conduct mixing at a temperature not lower than the phase transition temperature (Tc) of the lipid. Alternatively, should the carrier material produce micelles, the ingredients should be used in concentrations above their critical micelle concentration (CMC).

The particle size of the carrier complex can be controlled by the constituents and charge of the carrier material, the speed of the stirring and the shape of the reaction vessel. Thus when a comparatively mild mixing is performed, the particle sizes of the carrier complexes tend to become relatively bigger. When a high speed mixing is performed the particle sizes tend to be smaller. Moreover, for the purpose of controlling the particle size distribution of the carrier complexes, it is possible to pass the particles through a filter such as an ultra filtration unit, which may employ a polycarbonate membrane filter for example. Preferably, if ultra filtration is used in conjunction with the present invention, it generally has a pore size of 500 nm or less. When the carrier complex is produced when filtered through filters of 400 nm pore size or less, there is no need to perform any further sterilization process. Hence, sterile carrier complexes can be produced using a simple protocol within as little as 30 minutes.

Incorporation of heat-sensitive bioactive ingredients (such as DNA, RNA) to the carrier complexes can be introduced to the complexation zone when the temperature is reduced (e.g. after 30 minutes heating) to an acceptable temperature (e.g. 40° C.). In order to assist in the formulation of a stable carrier complex, the carrier material(s) may be selected for a given bioactive ingredient on the basis of its charge. Therefore, the binding, encapsulation, entrapment or incorporation of these bioactive ingredients to the carrier complexes can be achieved by using oppositely charged carrier complexes, for example, anionic or cationic polymer- or lipid-based complexes. The bioactive ingredients can even be incorporated to similarly charged carrier complexes by the mediation of divalent metal cations such as Ca²⁺ or Mg²⁺. High drug entrapment efficiencies can be achieved by use of carrier complex components having a charge opposite to the charge of the bioactive ingredient or drug. When the amount of the bioactive ingredient is not more than 50% on the ion equivalent basis relative to the oppositely charged carrier complex or, more precisely, relative to the charged molecules involved in charging of the carrier complex, a higher bioactive ingredient encapsulation efficiency can be attained.

Preferably, the method is performed in a vessel with a stirring unit disposed in its interior. The vessel may additionally comprise a heating element.

The heating element of the illustrated vessel is a separate unit. In case of small, lab scale vessel (e.g. up to 2 Lt volume) a normal, laboratory “hot-plate stirrer” can be used. In the case of large, industrial-scale vessels, they are manufactured with two walls providing a “heating jacket” through which hot water or steam can adjust the temperature inside the vessel to a desired level.

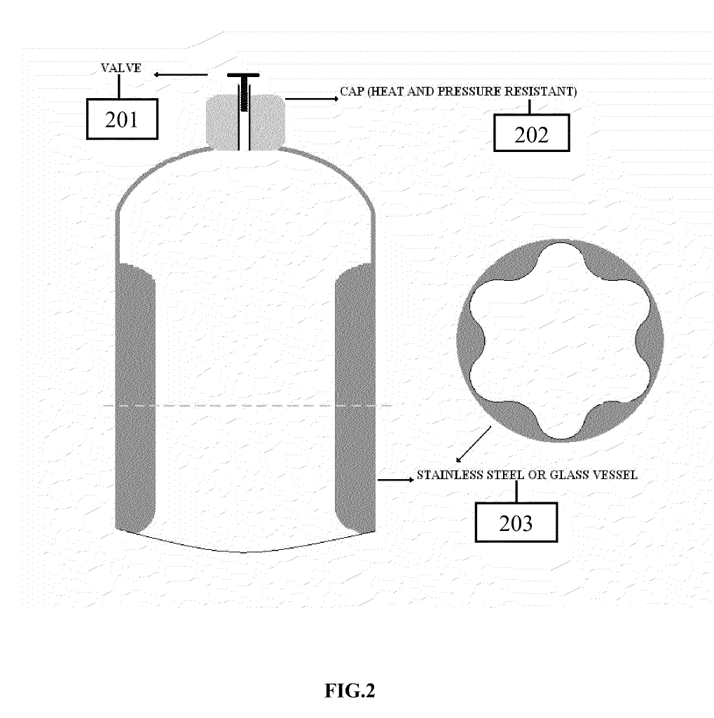

Such a vessel may be manufactured from stainless steel or heat resistant glass with or without Teflon coatings. Furthermore, the vessel can have an interior with an undulating cross-section defining a plurality of baffles (FIG. 2). The vessel may also be charged with an aqueous medium. The aqueous medium may comprise a carrier material and additionally the bioactive ingredient and auxiliary materials such as membrane stabilizers, charge modifiers and antioxidants.

Stirring: In case of small, lab-scale vessel (e.g. up to 2 Lt volume), magnet bars along with a normal, laboratory “hot-plate stirrer”, or overhead stirrers can be used to stir the contents. In the case of large, industrial-scale vessels, propellers embedded inside the vessel, or overhead stirrers are used for this purpose.

Addition of bioactive: The bioactive material such as drug, can be added in several stages and this provides the present invention a desirable flexibility to allow the encapsulation/entrapment of vast range of drugs and other bioactive agents. The bioactive agents:

i) can be added initially along with the components of the carrier system and the suspending medium (i.e. aqueous medium);
ii) can be added after the stirring has been started (i.e. incorporated into step (b), see below);
iii) can be added after termination of the “stirring and heating” step i.e. after the carrier system has completely formed (Please see Example 8).

Recovering step: Final products, according to the present invention, are recovered from the vessel as they are, with no need of further process. However, they can be subject to filtration, sterilization or other processes if required.

According to another embodiment of the present invention, a fast and efficient method, wherein the method does not require the carrier materials to be hydrated separately prior to heating.

According to one embodiment, the method does not require volatile organic solvents to be used in the manufacture of carrier complexes.

The embodiments herein are related to a method of producing a carrier complex of a bioactive ingredient disposed within a carrier material that involves providing a complexation zone supplied with an aqueous medium containing the carrier material; simultaneously stirring and heating the aqueous medium; adding the bioactive ingredient to the aqueous medium and maintaining the complexation zone and recovering from the complexation zone a carrier complex.

The most specific substance that is focused on in this invention is a vessel with a stirring unit disposed in its interior, wherein the method of producing a carrier complex is carried out. The vessel may additionally comprise a heating element, wherein the heating element is a separate unit in the illustrated vessel.

According to another embodiment, storing the final product under optimum conditions, such as avoiding light exposure and at ideal temperatures and under an inert atmosphere, such as nitrogen or argon, as appropriate.

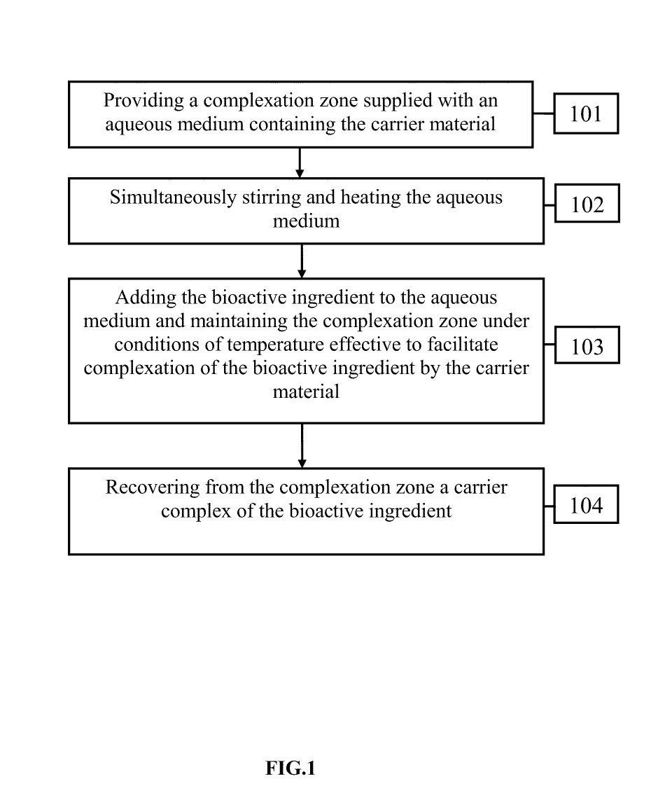

FIG. 1 illustrates a flow chart explaining a method of producing a carrier system for the encapsulation or entrapment of a bioactive agent according to one embodiment of the embodiments herein. With respect to FIG. 1, a method of producing a carrier complex of a bioactive ingredient, involves (a) providing a complexation zone supplied with an aqueous medium containing the carrier material (101); (b) simultaneously stirring and heating the aqueous medium (102); (c) adding the bioactive ingredient to the aqueous medium and maintaining the complexation zone under conditions of temperature effective to facilitate complexation of the bioactive ingredient by the carrier material (103); and (d) recovering from the complexation zone a carrier complex of the bioactive ingredient (104).

FIG. 2 illustrates a schematic representation of a reaction vessel used for the manufacture of carrier complexes. With respect to FIG. 2, the vessel comprising of a valve (201) at the top of the vessel for regulating the flow of fluids, further comprising a heat and pressure resistant cap (202) and may additionally comprise a heating element. Such a vessel may be manufactured from stainless steel or heat resistant glass (203) with or without Teflon coatings. Furthermore, the vessel can have an interior with an undulating cross-section defining a plurality of baffles (FIG. 2). The vessel may also be charged with an aqueous medium. The aqueous medium may comprise a carrier material and additionally the bioactive ingredient and auxiliary materials such as membrane stabilizers, charge modifiers and antioxidants.

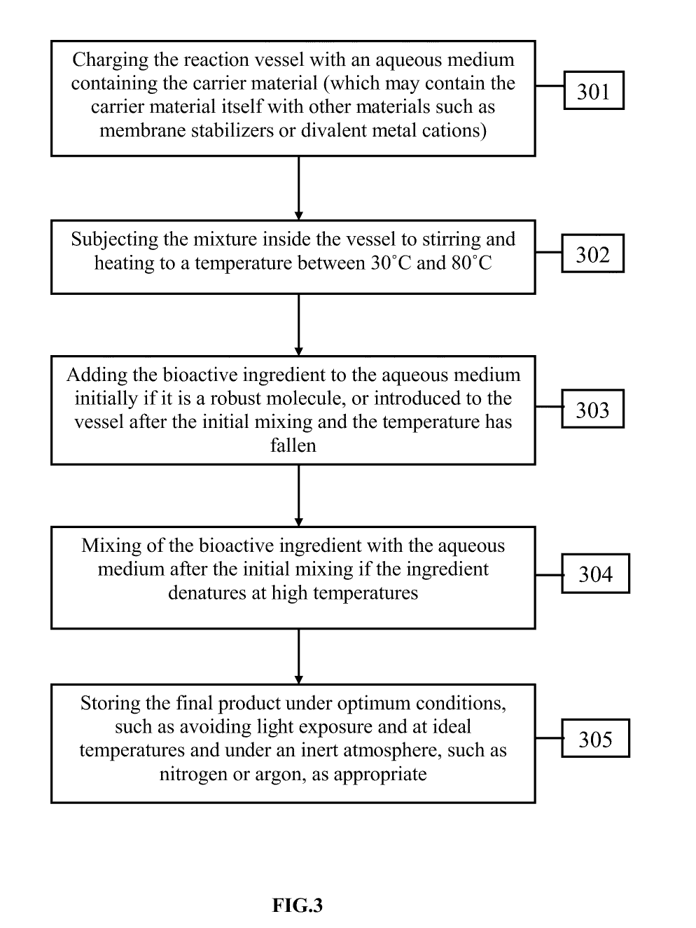

FIG. 3 illustrates the basic protocol of the method of producing carrier complexes. With respect to FIG. 3, charging the reaction vessel with an aqueous medium containing the carrier material (which may contain the carrier material itself with other materials such as membrane stabilizers or divalent metal cations) (301). Subjecting the mixture inside the vessel to stirring and heating to a temperature between 30° C. and 80° C. (302). Adding the bioactive ingredient to the aqueous medium initially if it is a robust molecule, or introduced to the vessel after the initial mixing and the temperature has fallen (303). Mixing of the bioactive ingredient with the aqueous medium after the initial mixing if the ingredient denatures at high temperatures (304). Storing the final product under optimum conditions, such as avoiding light exposure and at ideal temperatures and under an inert atmosphere, such as nitrogen or argon, as appropriate (305).

### EXPERIMENTAL DATA

The present invention will now be described by way of examples:

### Example 1

This example illustrates the basic protocol of the method of producing carrier complexes.

A reaction vessel is charged with an aqueous medium, which contains the carrier material (which may contain the carrier material itself with other materials such as membrane stabilizers or divalent metal cations). The mixture inside the vessel will be subjected to stirring and heating to a temperature between 30° C. and 80° C. The bioactive ingredient (which may be a pharmaceutical compound for example) can be added to the aqueous medium initially if it is a robust molecule, or introduced to the vessel after the initial mixing and the temperature has fallen. The bioactive ingredient may be mixed with the aqueous medium after the initial mixing if the ingredient is for example an enzyme, which may denature at high temperatures. The final product should be stored under optimum conditions, such as avoiding light exposure and at ideal temperatures and under an inert atmosphere, such as nitrogen or argon, as appropriate.

### Example 2

An experiment was conducted to produce a carrier complex incorporating 5-fluorouracil in a dipalmitoylphosphatidylcholine: dicetylphosphate (DPPC:DCP) carrier material. DPPC:DCP (7:3 molar ratio), containing 10 mM to 50 mM of total lipid, was mixed with 5-fluorouracil (at a concentration of 400 mM) and phosphate buffered saline (pH: 7.4) in a reaction vessel under continuous heating (50 to 70° C.) conditions, and whilst being stirred continuously. The reaction volume can be between 5 ml and 1000 liters. Carrier complexes were formed within 30 to 60 minutes with high yield, narrow size distribution and high 5-fluorouracil incorporation efficiency. Formation of carrier complexes are attested by microscopic visualisation, size distributions are measured by light scattering and release behaviour studies are performed in buffer or bodily fluids such as plasma/serum, urine, salivary fluid, and bronchoalveolar lavage.

### Example 3

Carrier complexes were formed according to the same protocol as described in Example 2, but with isotonic HEPES buffer (pH: 7.4) in place of phosphate buffered saline with similar results.

### Example 4

Carrier complexes were formed according to the same protocol as described in Example 2, but with isotonic sucrose solution (9.25% w/v sucrose in distilled water) in place of phosphate buffered saline with similar results.

### Example 5

Carrier complexes were formed according to the same protocol as described in Examples 2 to 4 but with stearylamine (cationic lipid) in place of DCP (anionic lipid) and producing similar results.

### Example 6

Carrier complexes were formed according to the same protocol as described in Examples 2 to 5 but with glutathione in place of 5-fluorouracil.

### Example 7

Carrier complexes were formed according to Examples 2 to 5 but with PEG-PE (PEG: 2000) in place of DPPC:DCP at a temperature of 60 to 80° C.

### Example 8

Carrier complexes were formed according to Examples 2 to 5 but with no drug. To these ‘empty’ carrier complexes in the reaction vessel, and at a temperature not more than 40° C., plasmid DNA (pcDNA3.1/His B/lacZ, 8578 nucleotides) (Invitrogen) and then calcium chloride (50 mM) are added followed by the incubation of the mixture for 15 to 30 minutes. Characterisation of the carrier complex were performed as explained above while transfection efficiency was evaluated by using appropriate cell culture systems such as human respiratory epithelial cells (16HBE14o-).

### Example 9

Carrier complexes were formed according to the same protocol as in Examples 2 to 5 but with a composition of Span-60 instead of DPPC at a temperature of 40 to 70° C.

### Example 10

Carrier complexes were formed according to the same protocol as outlined in Examples 2 to 5 but with a composition of Tween 80 in place of DPPC at a temperature of 40 to 70° C.

### Example 11

An experiment was conducted to produce a carrier complex incorporating the bioactive pharmaceutical of paclitaxel ingredient surrounded by a carrier material of a PDLLA-PEG copolymer.

Five mg/ml of PDLLA-PEG copolymer (number average molecular weight of PDLLA: 1866, number average molecular weight of PEG: 3300-4000) was mixed with paclitaxel (at a concentration of 2 to 10% w/w) and phosphate buffer (pH: 7.4) in the reaction vessel (as described above in Example 1) whilst being heated (60 to 80° C.) and stirred continuously.

The reaction volume can for example be as small as 5 ml, or as large as 1000 liters, using industrial-sized equipment. The carrier complexes are formed within 30 to 60 minutes with high yield, narrow size distribution and high paclitaxel incorporation efficiency. Formation of carrier complexes are attested by microscopic visualisation, size distributions are measured by light scattering and release behaviour studies are performed in buffer or bodily fluids such as plasma/serum, urine, salivary fluid, and bronchoalveolar lavage fluid.

### Example 12

A further experiment was conducted to produce a carrier complex incorporating the drug daunorubicin by a carrier material of a PNIPAAm-b-PEG copolymer. One mg/ml of PNIPAAm-b-PEG copolymer (24300:5000 g/mol) was mixed with daunorubicin (at a concentration of 2 mg/ml) and phosphate buffer (pH: 7.4) in a reaction vessel, which was heated continuously at a temperature between 60 to 80° C., whilst being stirred. The reaction volume as in Example 2 can for example be as small as 5 ml, or as large as 1000 liters. Carrier complex produced using this method is formed within 30 to 60 minutes with a high yield, narrow size distribution and high daunorubicin incorporation efficiency. Again, as with Example 11, formation of carrier complexes are attested by microscopic visualization, size distributions are measured by light scattering and release behaviour studies are performed in buffer or bodily fluids such as plasma/serum, urine, salivary fluid, and bronchoalveolar lavage.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A method of producing a carrier complex of a bioactive ingredient disposed within a carrier material, the method consisting the steps of:
providing a complexation zone supplied with an aqueous medium containing the carrier material;
stirring and heating the aqueous medium simultaneously;
adding the bioactive ingredient to the aqueous medium and maintaining the complexation zone under conditions of temperature to facilitate complexation of the bioactive ingredient by the carrier material; and
recovering a carrier complex of the bioactive ingredient from the complexation zone.

2. The method according to claim 1, wherein the step of adding of the bioactive ingredient to the aqueous medium is simultaneously included with the complexation zone supplied with the aqueous medium containing the carrier material.

3. The method according to claim 1, wherein the step of adding of the bioactive ingredient to the aqueous medium is simultaneously subjected to simultaneous stirring and heating of the aqueous medium.

4. The method according to claim 1, wherein the aqueous medium is heated to a temperature in the range of 30° C. to 80° C. during the step involving the stirring and heating and/or in the step of adding the bioactive ingredient to the aqueous medium and maintaining the complexation zone under conditions of temperature to facilitate complexation of the bioactive ingredient by the carrier material.

5. The method according to claims 1, wherein the temperature of the aqueous medium during the addition of the bioactive ingredient to the aqueous medium and maintaining the complexation zone to facilitate complexation of the bioactive ingredient by the carrier material is lesser than the temperature of the aqueous medium during the stirring and heating.

6. The method as claimed in claim 5, wherein the temperature is in the range of 40° C. to 60° C.

7. The method according to claim 1, wherein the duration of the method of producing a carrier complex of a bioactive ingredient disposed within a carrier material is 2 hours.

8. The method according to claim 1, wherein the carrier complex comprises a niosome, a micelle, a microsphere, a nanosphere, a nanoparticle, a liposome, a nanoliposome, a vesicular phospholipid gel, an archaeosome or a cochleate.

9. The method according to claim 1, wherein the carrier material is selected from one or more elements from the group comprising a copolymer, a synthetic polymer-lipid conjugate, a lipid, a phospholipid or a non-ionic surfactant.

10. The method according to claim 9, wherein the carrier material comprising of the lipids, the temperature of the aqueous medium in the step of stirring and heating is more effective to conduct mixing at a temperature not lower than the phase transition temperature of the lipid.

11. The method according to claim 9, wherein the carrier material further comprising of one or more auxiliary materials.

12. The method according to claim 11, wherein the auxiliary material is selected from one or more elements from the group comprising membrane stabilizers, charge modifiers, divalent cations and antioxidants.

13. The method according to claim 12, wherein the membrane stabilizer is selected from one or more elements from the group comprising a charge modifier or inducer.

14. The method according to claim 13, wherein the charge modifiers or inducers are selected from one or more elements from the group comprising dicetylphosphate, phosphatidic acid, stearylamine, ganglioside or the like.

15. The method according to claim 12, wherein the antioxidant is selected from one or more elements from the group comprising a-tocopherol, any of the group tocopheryl phosphates, or the like.

16. The method according to claim 9, wherein the carrier material produces micelles provided the concentrations of carrier material are above their critical micelle concentration.

17. The method according to claim 13, wherein proportion of the membrane stabilizers is in the range of 0.1-30% molar ratio of the carrier material.

18. The method according to claim 13, wherein the proportion of membrane stabilizers is in the preferable range of 5-20% molar ratio of the membrane components.

19. The method according to claim 14, wherein the proportion of charge modifiers is in the range of 0.1-50% molar ratio of the carrier material.

20. The method according to claim 14, wherein the proportion of charge modifiers is in the preferable range of 10-30% molar ratio of the membrane components.

21. The method according to claim 1, wherein the aqueous medium is selected from one or more elements from a group comprising water, distilled water or Milli-Q reagent grade water, a physiological saline solution, a buffer solution or an aqueous carbohydrate solution or may be a mixture thereof.
